(19) United States
(12) Patent Application Publication
(10) Pub. No.: US 2005/0228223 A1
(43) Pub. Date: Oct. 13, 2005

(54) Method for obtaining medically relevant data from the gastrointestinal tract of a human or an animal and measuring probe suitable therefor

(76) Inventors: Klaus Abraham-Fuchs, Erlangen (DE); Rainer Kuth, Herzogenaurach (DE); Eva Rumpel, Erlangen (DE); Markus Schmidt, Nuernberg (DE); Siegfried Schneider, Erlangen (DE); Horst Schreiner, Fuerth (DE); Gudrun Zahlmann, Neumarkt (DE)

Correspondence Address: HARNESS, DICKEY & PIERCE, P.L.C., P.O.BOX 8910, RESTON, VA, 20195, US

(21) Appl. No.: 11/093,279

(22) Filed: Mar. 30, 2005

(30) Foreign Application Priority Data
Mar. 31, 2004 (DE) 10 2004 016 694.3

(51) Int. Cl. A61B 1/00

(52) U.S. Cl. 600/102

(57) ABSTRACT

A method is for obtaining medically relevant data from the gastrointestinal tract of a patient (human or an animal). The patient is orally administered with a transponder and signals emitted by the transponder during or after passage through the gastrointestinal tract are received, with the aid of a reading device for example. A measuring probe may be suitable for carrying out the method. The probe is designed as a transponder and includes a sensor unit for recording chemical, electrochemical or physical parameters.

The present application hereby claims priority under 35 U.S.C. §119 on German patent application number DE 10 2004 016 694.3 filed Mar. 31, 2004, the entire contents of which is hereby incorporated herein by reference.

### FIELD OF THE INVENTION

The invention generally relates to a method for obtaining medically relevant data from the gastrointestinal tract of a human or animal, and/or to a measuring probe suitable therefor. Data of the type in question may be, for example, physical, chemical and/or electrochemical parameters and/or measured values, which may for example be processed for diagnostic purposes.

### BACKGROUND OF THE INVENTION

In the past, such data have often been obtained by endoscopy. Endoscopic methods are generally very elaborate and are usually felt to be uncomfortable by patients.

US 2002/0198470 A1 discloses a device in the form of an indigestible capsule for diagnosis or treatment of the gastrointestinal tract of a human. The capsule passes through the gastrointestinal tract and has a locating system based on an acoustic signal and optionally a radio signal for reference.

GB 2 374 149 A discloses a device in the form of an indigestible capsule for medicament release in the gastrointestinal tract of a human. The capsule passes through the gastrointestinal tract and has a memory, in which data can be stored.

### SUMMARY OF THE INVENTION

It is an object of an embodiment of the invention to provide a method and/or a measuring probe suitable therefor, which can, for example, be used in a straightforward way that is comfortable for the patient.

An object may be achieved by a method and/or a measuring probe. A method of an embodiment may be one in which a transponder functioning as a measuring probe is orally administered to the patient and signals emitted thereby during or after passage through the gastrointestinal tract are received with the aid of a reading device. The signals or data which are read may then processed using data technology, and for example, stored in an electronic patient file. The transponder may, for example, be taken with liquid.

According to an embodiment of the invention, it is integrated in a preparation which is solid overall or at least has a solid shell, for instance a tablet, pill or a capsule, which is readily possible owing to the small size of modern transponders. In this form, it can be swallowed more easily and is protected against damage during administration, for instance in the case of animals. The solid preparation contains a pharmaceutical active agent and is intended to dissolve in the patient's gastrointestinal tract. A transponder, in an embodiment, suitable for carrying out a method of the aforementioned type may be furthermore encased by a biocompatible protective layer. The protective layer may include a material which is resistant to both gastric acid and digestive enzymes. For example, PVC (polyvinyl chloride) or POM (polyoxymethylene) are suitable.

The success of a medication treatment depends crucially on taking the medicament regularly. This may be a problem with dementive or psychologically disturbed patients. In this case, there is often no check as to whether the medicaments have actually been taken.

In order to carry out medical studies, it is generally necessary to have a sizeable number of subjects for reasons of significance. In order to prevent manipulation, suitable measures should be taken to monitor the medication during the trial period.

In a preferred variant of the method of an embodiment, this involves administering a patient/subject with a medicament in a solid preparation containing a transponder allocated to them using data technology. The allocation is carried out in a manner known per se using an ID number which is stored in the transponder and can be read by a reading device.

In the simplest embodiment, the transponder is only used to establish whether or not the patient has taken a medicament. To this end, it is sufficient for the transponder to emit signals that contain no further information besides the ID number. These signals may be recorded by a reading device carried on the body, so that it is possible to detect that the transponder is present and therefore that the medicament has been taken.

Naturally, the duration for which the transponder is present in the body may also be determined, for example so as to establish whether the residence time is too short, for instance in the event of diarrhea. In principle, the same may also be achieved by a reading device arranged away from the body, for example on a toilet.

Data of a chemical, electrochemical or physical nature can be obtained with the aid of a sensor unit integrated in the transponder. For example, it is feasible for temperature measurements to be taken continuously or at particular time intervals by a temperature sensor during the passage of the transponder through the gastrointestinal tract, for example with the aid of a thermocouple, in which case the temperature data can be recorded by a reading device. The readout may then likewise take place in particular time slots, optionally synchronized with the time intervals of the temperature measurement.

In this way, it is possible to compile a temperature scan over the entire length of the gastrointestinal tract. This, or even individual temperature measurements, are used to increase the security against manipulation in the case of clinical studies. In this context, a sensor unit which comprises a pH probe is also expedient. In this way, for example, it is possible to discover pathological deviations of the pH values prevailing in individual sections of the gastrointestinal tract.

In the event that medication is being monitored, it is thereby possible to establish whether the preparation has dissolved already in the stomach or later, for example in the duodenum. A pH measurement also increases the security against manipulation in clinical studies. Information about when the preparation has dissolved or the medicament has been released can be determined by determining the electrical conductivity of the medium surrounding the transponder.

Another way of detecting release of the medicament is to use a sensor which detects a particular ingredient of a medicament in the immediate vicinity of the transponder after release. In one embodiment, a transponder is allocated to a particular medicament. Usability of a transponder for any medicaments is achieved when the preparation is supplemented with a biocompatible substance detectable by a sensor contained in the transponder when it is released.

A transponder suitable for carrying out a method of at least one embodiment may be encased by a biocompatible protective layer transparent to radiofrequency electromagnetic radiation. For example, PVC (polyvinyl chloride) or POM (polyoxymethylene) are suitable. In cases when the transponder contains a sensor unit that requires direct fluidic contact with its immediate environment, the protective layer should be interrupted by a porous material which is in contact with the sensor unit. It may likewise be a stable material of the aforementioned type, although this will need to be permeated by pores.

Naturally, it is also possible for an active sensing area of the sensor unit to be arranged on the outside of the protective layer enclosing the transponder. In this case, there will be a connection to the sensor unit contained inside the protective layer, for example in the form of electrical cables or the like. In order for data obtained by the sensor unit to be temporarily stored at least until they have been read by the reading device, it is expedient for there to be an electronic memory in the transponder.

### BRIEF DESCRIPTION OF THE DRAWINGS

Embodiments of the invention will be described in more detail below with reference to the appended drawings, in which,

FIG. 1 shows a cross section through a capsule containing a transponder,

FIG. 2 shows a cross section through a transponder,

FIG. 3 shows a part of an alternatively configured transponder in a cross-sectional representation,

FIG. 4 shows a schematic representation showing the arrangement of a reading device on a toilet.

### DETAILED DESCRIPTION OF THE EXAMPLE EMBODIMENTS

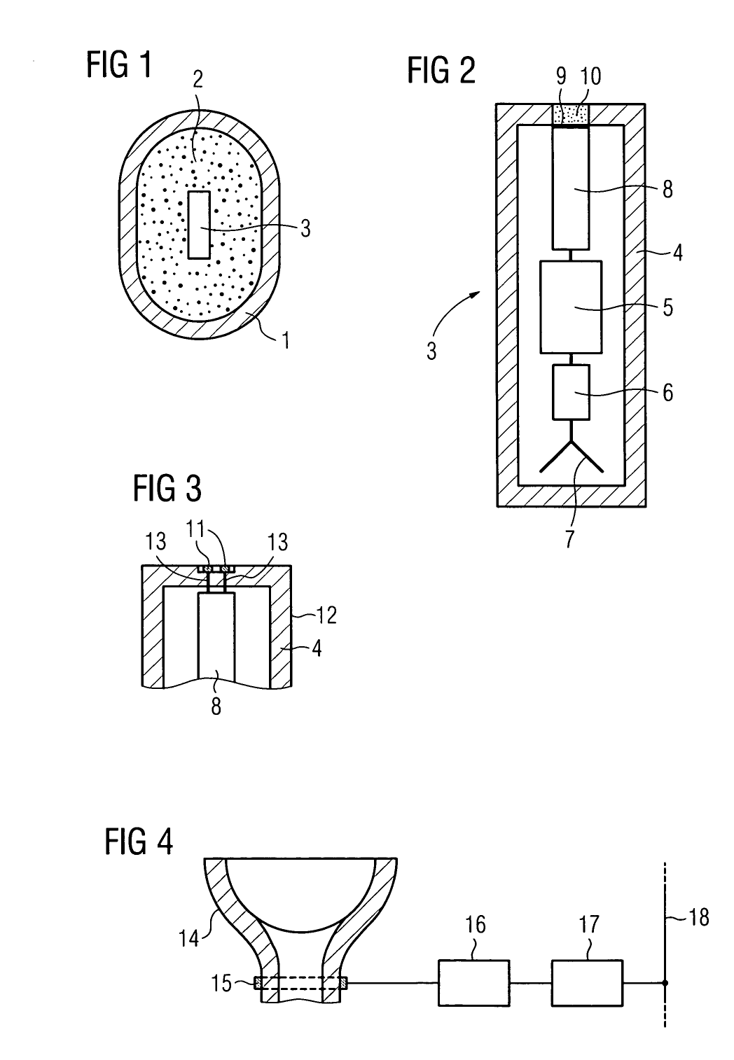

The preparation represented in FIG. 1 is a capsule of an embodiment of the invention. It contains a shell 1 that dissolves in the ambient conditions of the gastrointestinal tract, particularly of the stomach. It is used as a container for a medicament 2 and a transponder 3.

The transponder 3 is a structure shaped approximately like a tree cone, which is encased by a stable protective layer 4, such as PVC or POM for example. The transponder 3 contains a microprocessor 5, a quartz oscillator 6 and an antenna 7. These basic components may be supplemented by a sensor unit 8. The latter may be used to record physical parameters such as the temperature, chemical or electrochemical parameters such as the pH, the electrical conductivity or the concentration of particular chemical substances.

In cases when a fluidic connection is required between an active sensing area 9 of the sensor unit 8 and the immediate environment of the transponder 3, there should be a porous material 10 across the protective layer 4. This material is likewise selected so that it can withstand the chemical conditions of the gastrointestinal tract. It does, however, have pores which allow liquid to access the sensing area 9. Access of liquid into the interior of the transponder can be prevented by suitable sealing measures.

Another option is to arrange an active sensing area, for example electrodes 11, on the outside of the protective layer 4. In this case, suitable connections will pass through the latter—in the present case electrical wires 13. The wires 13 are embedded in the protective layer 4 in this embodiment, but not are enclosed by it.

Reading devices 16 used to read data from a transponder 3 may, as mentioned above, be carried directly by the patient on their body. In many cases, it is sufficient merely to establish that the transponder has left the gastrointestinal tract. This may be done by a receiving antenna 15 of a reading device 16 which is fitted to a device such as toilet for example, for instance in the vicinity of its seat or its outflow pipe 14. The reading device 16 may have a signal connection via a CPU to a network 18.

Exemplary embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

1. A method for obtaining medically relevant data from a gastrointestinal tract of a patient, comprising:
orally administering, in the patient, a transponder designed to monitor a medication, the transponder being enclosed by a biocompatible protective layer and being integrated in a preparation, the preparation being solid overall or having a solid shell, containing a pharmaceutical active agent, and being dissolvable in the patient's gastrointestinal tract; and
receiving signals emitted from the transponder during or after passage through the gastrointestinal tract.

2. The method as claimed in claim 1, wherein at least one of chemical, electrochemical and physical parameters of the gastrointestinal tract are determined with the aid of a sensor unit, integrated in the transponder.

3. The method as claimed in claim 2, wherein the pH of the gastrointestinal tract is determined with the aid of a pH probe integrated in the sensor unit.

4. The method as claimed in claim 3, wherein pathological deviations of pH values prevailing in individual sections of the gastrointestinal tract are detected with the aid of the pH probe.

5. The method as claimed in claim 3, wherein the pH probe is used to establish, for the medication, whether the preparation dissolves in a first section, or in a subsequent section.

6. The method as claimed in claim 2, wherein a biocompatible substrate added to the preparation is detected with the aid of the sensor unit in order to monitor at least one of the dissolving of the preparation and the release of a medicament in the gastrointestinal tract.

7. The method as claimed in claim 1, wherein the transponder is read out by a reading device carried by the patient on their body.

8. A transponder for monitoring the medication of a patient, the transponder being enclosed by a biocompatible protective layer and integrated in a preparation which is solid overall or has a solid shell, the preparation containing a pharmaceutical active agent and being dissolvable in a gastrointestinal tract of the patient after oral administration to the patient, the transponder being adapted to emit signals, receivable by at least one reading device.

9. The transponder as claimed in claim 8, wherein the transponder comprises a sensor unit for recording at least one of chemical, electrochemical and physical parameters.

10. The transponder as claimed in claim 8, wherein the transponder is allocated to the patient using data technology.

11. The transponder as claimed in claim 9, wherein the reading device is arrangeable on a toilet.

12. The transponder as claimed in claim 11, wherein the sensor unit comprises a pH probe.

13. The transponder as claimed in claim 12, wherein the pH probe is adapted to detect pathological deviations of pH values prevailing in individual sections of the gastrointestinal tract.

14. The transponder as claimed in claim 12, wherein the pH probe is adapted to establish for the medication, whether the preparation dissolves in a first section or in a subsequent section.

15. The transponder as claimed in claim 11, wherein the biocompatible protective layer is transparent to radiofrequency electromagnetic radiation.

16. The transponder as claimed in claim 15, further comprising a porous material, in contact with an active sensing region of the sensor unit, across the protective layer.

17. The transponder as claimed in claim 11, further comprising an electronic memory.

18. A pharmaceutical active agent which is integrated in a solid preparation, wherein a transponder as claimed in claim 8 is added to the active agent.

19. The method of claim 5, wherein the first section is the stomach, and the subsequent section is the duodenum.

20. The method as claimed in claim 4, wherein the pH probe is used to establish, for the medication, whether the preparation dissolves in a first section, or in a subsequent section.

21. The method of claim 20, wherein the first section is the stomach, and the subsequent section is the duodenum.

22. The transponder as claimed in claim 13, wherein the pH probe is adapted to establish for the medication, whether the preparation dissolves in a first section or in a subsequent section.

23. The transponder as claimed in claim 14, wherein the first section is the stomach, and the subsequent section is the duodenum.

24. The transponder as claimed in claim 22, wherein the first section is the stomach, and the subsequent section is the duodenum.

25. A transponder for monitoring the medication of a patient, the transponder being enclosed by a biocompatible protective layer and integrated in a preparation which at least includes a solid shell, the preparation containing a pharmaceutical active agent and being dissolvable in a gastrointestinal tract of the patient after oral administration to the patient, the transponder including a sensor unit for recording at least one of chemical, electrochemical and physical parameters.

26. The transponder as claimed in claim 25, wherein the transponder is adapted to emit signals, receivable by at least one reading device.
